(19) United States
(12) Patent Application Publication
(10) Pub. No.: US 2026/0183763 A1
(43) Pub. Date: Jul. 2, 2026

(54) ELECTROKINETIC MICROFLUIDIC CONCENTRATOR CHIP DEVICE AND METHOD OF USE

(71) Applicant: New York University in Abu Dhabi Corporation, Abu Dhabi (AE)

(72) Inventors: Yong-Ak Song, Abu Dhabi (AE); Jongmin Kim, Abu Dhabi (AE); Ajymurat Orozaliev, Abu Dhabi (AE); Sarah Sahloul, Abu Dhabi (AE); Ibrahim Chehade, Mississauga (CA); Mohamed Al-Sayegh, Abu Dhabi (AE)

(21) Appl. No.: 18/867,991

(22) PCT Filed: May 26, 2023
(86) PCT No.: PCT/IB2023/000301

§ 371 Date: Nov. 21, 2024

Related U.S. Application Data
(60) Provisional application No. 63/346,082, filed on May 26, 2022.

(51) Int. Cl. B01L 3/00; C12Q 1/44; C12Q 1/70; G01N 1/40

(52) U.S. Cl. CPC B01L 3/502753; C12Q 1/44; C12Q 1/70; G01N 1/40; B01L 2200/10; B01L 2200/16; B01L 2300/0645; B01L 2300/12; G01N 2001/4038; G01N 2333/165

(57) ABSTRACT

Aspects of the present invention relate to a microfluidic concentrator chip device including a microfluidic chip having a flat body with a top and bottom surface, a first end, a second end, and a length therebetween, a channel in the body of the chip, extending from near the first end of the chip to near the second end of the chip, the channel comprising a bottom surface, a top surface, sidewalls extending from the bottom surface to the top surface, and a first end, a second end, and a length therebetween, a hydrogel membrane having a height and a width positioned within the channel, a buffer solution contained within the channel, a first electrode positioned near the first end of the channel in fluid communication with the buffer solution, a second electrode positioned near the second end of the channel in fluid communication with the buffer solution, and a power supply connected to the first and second electrodes, configured to apply a voltage across the channel.

### CROSS-REFERENCE TO RELATED APPLICATIONS

This application claims priority to U.S. Provisional Patent Application No. 63/346,082, filed May 26, 2022, the contents of which are incorporated by reference herein in its entirety.

### BACKGROUND OF THE INVENTION

Since the beginning of the pandemic from COVID-19, the outbreak of SARS-COV-2 has led to more than 196 million infections and more than 4 million deaths as of the middle of 2021, as reported by World Health Organization. Detection and diagnosis of SARS-COV-2 has created a large stressor for healthcare systems and personnel. In general, polymerase chain reaction (PCR) and reverse transcription polymerase chain reaction (RT-PCR) are widely used as a detection approach for SARS-COV-2, which often requires sophisticated protocols consisting of multiple steps. These protocols often result in cross contamination between each step and thereby biased detection results. In addition, it takes 1-4 hours for detection of SARS-COV-2 using traditional tests, which retards identification of infected patients and reduces control of disease spread. Therefore, a rapid, early-stage screening detection platform is crucial to effectively diagnose the rapid transmission of SARS-COV-2, and other diseases.

Thus, there is a need in the art for a rapid early-stage screening detection platform for the diagnosis of disease. The present invention satisfies that need.

### SUMMARY OF THE INVENTION

Aspects of the present invention relate to a microfluidic concentrator chip device including a microfluidic chip having a flat body with a top and bottom surface, a first end, a second end, and a length therebetween, a channel in the body of the chip, extending from near the first end of the chip to near the second end of the chip, the channel comprising a bottom surface, a top surface, sidewalls extending from the bottom surface to the top surface, and a first end, a second end, and a length therebetween, a hydrogel membrane having a height and a width positioned within the channel, a buffer solution contained within the channel, a first electrode positioned near the first end of the channel in fluid communication with the buffer solution, a second electrode positioned near the second end of the channel in fluid communication with the buffer solution, and a power supply connected to the first and second electrodes, configured to apply a voltage across the channel.

In some embodiments, the hydrogel membrane comprises a negatively charged sulfonate group. In some embodiments, the hydrogel membrane comprises polymerized 2-Acrylamido-2-methylpropane sulfonic acid. In some embodiments, the hydrogel membrane comprises N,N′-Methylenebisacrylamide as a crosslinker. In some embodiments, the buffer comprises any of Cas12a, Cas13a, a reaction buffer, a ribonuclease inhibitor, and a fluorescent reporter reagent. In some embodiments, the hydrogel membrane has a height that is between 25-40% of the height of the sidewalls of the channel. In some embodiments, the device includes one or more pillars extending downwards from the top surface of the channel, wherein the pillars pass through at least a portion of the hydrogel membrane, thereby anchoring the hydrogel membrane in the channel.

In some aspects, the present invention provides a method of detecting a target analyte in a sample on a microfluidic concentrator chip, the method having the steps of a) heating a test sample to a set temperature for a period of time, b) flowing a test sample through a channel of a microfluidic concentrator chip wherein the test sample comprises a detectable molecule for detecting the presence of a target analyte in the test sample, c) applying a direct current (DC) voltage across the chip to generate an ion concentration polarization (ICP) region on the microfluidic concentrator chip, and d) detecting the detectable molecule in the ICP region, wherein an increase in the level of the detectable molecule in the ICP region as compared to a control level indicates the presence of the target molecule in the test sample.

In some embodiments, the test sample comprises a biological fluid sample selected from the group consisting of a saliva sample, a blood sample, a plasma sample and a serum sample. In some embodiments, the method further has the steps of a) flowing a test sample through a channel of a microfluidic concentrator chip wherein the test sample comprises a Cas protein at least one gRNA molecule, a ribonuclease inhibitor and a detectable molecule for detecting the presence of a target nucleic acid molecule in the test sample, b) applying a DC voltage across the chip to generate an ICP region on the microfluidic concentrator chip, and c) detecting the detectable molecule in the ICP region, wherein an increase in the level of the detectable molecule in the ICP region as compared to a control level indicates the presence of a target nucleic acid molecule in the test sample.

In some embodiments, the Cas protein is selected from the group consisting of a Cas12a and Cas13a protein. In some embodiments, the detectable molecule is a fluorophore quencher (FQ)-labeled reporter molecule. In some embodiments, the gRNA molecule is specific for binding to a viral RNA molecule. In some embodiments, the viral RNA molecule is a SARS-CoV-2 viral RNA molecule, and further comprising the step of indicating a SARS-COV-2 viral infection in a subject when the target nucleic acid molecule is detected in the test sample.

In some aspects, the present invention provides a method of concentrating an analyte in a sample having the steps of a) introducing a fluid to an inlet of the channel in any microfluidic concentrator chip device of the present invention, and b) applying a DC voltage across the chip to generate an ICP region in the channel, until the analyte is concentrated within the ICP region.

In some embodiments, the method further has the step of releasing the concentrated analyte containing fluid. In some embodiments, the method further has the step of applying the concentrated analyte containing fluid to a downstream assay for detection of the target analyte. In some embodiments, the downstream assay is selected from the group consisting of an immunoassay, a microarray, DNA hybridization assay, morpholino assay, aptamer assay, peptide nucleic acid (PNA) assay, and a target analyte detection assay.

In some embodiments, the method further has the step of applying the concentrated analyte containing fluid to a downstream detector for detection of the target analyte. In some embodiments, the downstream detector is selected from the group selected from a fluorescence detector, an electrochemical detector, a mass spectrometer, optical microring resonator, surface plasmon resonance (SPR) detector and a conductivity detector. In some embodiments, the target analyte is selected from the group consisting of a pathogen, a soluble antigen, a nucleic acid molecule, a toxin, a chemical, a bacterium, and a virus.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 depicts a perspective view of an exemplary microfluidic concentrator chip device according to aspects of the present invention.

FIG. 2. depicts a perspective view (bottom) of an exemplary microfluidic concentrator chip device with an enlarged top down view (top) of an exemplary hydrogel membrane according to aspects of the present invention.

FIG. 3. depicts a top down view of the loading and assay of an exemplary microfluidic concentrator chip device according to aspects of the present invention.

FIG. 4 is a series of drawings depicting an exemplary method of fabricating an exemplary microfluidic concentrator chip device according to aspects of the present invention.

FIG. 5A through FIG. 5D show the experimental data demonstrating one-step CRISPR-Cas detection of synthetic SARS-COV-2 cDNA by using the disclosed microfluidic concentrator chip device with ion concentration polarization (ICP). FIG. 5A shows a top down view of the microfluidic concentrator chip device with an enlarged view of a hydrogel membrane positioned in a channel. The region of interest (ROI) is highlighted with the yellow dotted line. FIG. 5B shows the results for time sequence images within 90 sec. showing an increased fluorescence signal intensity which implies successful detection of the synthetic SARS-COV-2 cDNA (10⁵ copies/μl) using the disclosed microfluidic chip device. FIG. 5C shows quantitatively analysed fluorescence signal intensity values from the various concentrations of SARS-COV-2 cDNA from 1 copy/μl to 10⁵ copies/μl within 300 sec. FIG. 5D shows the measurement of the initial slopes of the CRISPR-Cas detection for synthetic SARS-COV-2 cDNA within the first 90 seconds showing that even a cDNA concentration of 1 copy/μl can successfully be detected without prior PCR amplification using the disclosed microfluidic concentrator device.

FIG. 6A through FIG. 6C show experimental data demonstrating one-step CRISPR-Cas detection of synthetic SARS-COV-2 RNA by using the disclosed microfluidic concentrator chip device with ion concentration polarization (ICP). FIG. 6A shows time sequence images within 120 sec. showing increased fluorescence signal, which implies successful detection of the synthetic SARS-COV-2 RNA (10³ copies/μl) using the disclosed microfluidic chip. FIG. 6B shows the fluorescence intensity as a function of time indicating successful detection of the synthetic SARS-COV-2 RNA with various copies numbers in the disclosed microfluidic concentrator chip device. FIG. 6C shows the initial slopes of the CRISPR-Cas detection for synthetic SARS-COV-2 RNA demonstrating that the RNA concentration down to 1 copy/μl can be detected without PCR amplification and reverse transcription in the disclosed microfluidic concentrator device.

FIG. 7 shows exemplary experimental data demonstrating one-step CRISPR-Cas detection of SARS-COV-2 cDNA from patient samples using the disclosed microfluidic chip device with ICP. The slopes of the CRISPR-Cas detection were obtained from 12 positive patient samples and the LOD (blue dash line) is the slope obtained from 5 healthy samples as a reference.

FIGS. 8A and 8B show the results for the assessment and reproducibility of the one-step CRISPR-Cas detection of SARS-COV-2 RNA using the integrated microfluidic concentrator chip with ion concentration polarization (ICP). The fluorescence intensity was measured as a function of time, demonstrating successful and reproducible detection of the SARS-COV-2 RNA. The concentration of the SARS-COV-2 RNA ranged from 10⁻¹ to 10⁵ copies/μL. In each graph, the bold line represents the range used for linear regression analysis, which allows for accurate quantification and assessment of the detection performance.

FIGS. 9A and 9B show additional quantification of the signal obtained from the detection of SARS-COV-2. FIG. 9A shows the maximum intensity was obtained from the entire intensity values, showcasing the robust signal obtained from the positive samples. The quantified signal intensity clearly demonstrates the distinct detection of the positive samples in comparison to the control. FIG. 9B shows the initial slope of the signal from the SARS-COV-2 RNA is analyzed using linear regression. This analysis further confirms the clear differentiation between the positive samples and the control.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

“Amplification,” as used herein, refers to any in vitro process for increasing the number of copies of a nucleotide sequence or sequences, i.e., creating an amplification product which may include, by way of example additional target molecules, or target-like molecules or molecules complementary to the target molecule, which molecules are created by virtue of the presence of the target molecule in the sample. These amplification processes include but are not limited to polymerase chain reaction (PCR), multiplex PCR, Rolling Circle PCR, ligase chain reaction (LCR), loop-mediated isothermal amplification (LAMP) and recombinase polymerase amplification (RPA), and the like, in a situation where the target is a nucleic acid, an amplification product can be made enzymatically with DNA or RNA polymerases or transcriptases. Nucleic acid amplification results in the incorporation of nucleotides into DNA or RNA. As used herein, one amplification reaction may consist of many rounds of DNA replication. PCR is an example of a suitable method for DNA amplification. For example, one PCR reaction may consist of 2-40 “cycles” of denaturation and replication.

Any DNA sample may be used in practicing the present invention, including without limitation eukaryotic, prokaryotic, viral DNA, non-natural DNA, cDNA, and recombinant DNA molecules.

“Amplification products,” “amplified products” “PCR products” or “amplicons” comprise copies of the target sequence and are generated by hybridization and extension of an amplification primer. This term refers to both single stranded and double stranded amplification primer extension products which contain a copy of the original target sequence, including intermediates of the amplification reaction.

As used herein, an “antibody” encompasses naturally occurring immunoglobulins, fragments thereof, as well as non-naturally occurring immunoglobulins, including, for example, single chain antibodies, chimeric antibodies (e.g., humanized murine antibodies), heteroconjugate antibodies (e.g., bispecific antibodies). Fragments of antibodies include those that bind antigen, (e.g., Fab′, F(ab′)2, Fab, Fv, and rlgG). See, e.g., Pierce Catalog and Handbook, 1994-1995 (Pierce Chemical Co., Rockford, III); Kuby, J., Immunology, 3rd Ed., W.H. Freeman & Co., New York (1998). The term “antibody” further includes both polyclonal and monoclonal antibodies.

“Appropriate hybridization conditions” as used herein may mean conditions under which a first nucleic acid sequence (e.g., primer, etc.) will hybridize to a second nucleic acid sequence (e.g., target, etc.), such as, for example, in a complex mixture of nucleic acids. Appropriate hybridization conditions are sequence-dependent and will be different in different circumstances.

In some embodiments, an appropriate hybridization conditions may be selective or specific wherein a condition is selected to be about 5-10° C. lower than the thermal melting point (Tm) for the specific sequence at a defined ionic strength and pH. In some embodiments, an appropriate hybridization condition encompasses hybridization that occurs over a range of temperatures from more to less stringent. In some embodiments, a hybridization range may encompass hybridization that occurs from 98° C. to 10° C. According to the invention, such a hybridization range may be used to allow hybridization of the primers of the invention to target sequences with reduced specificity, for the purposes of amplifying a broad range of nucleic acid molecules with a single set of primers.

The term “control or reference standard” describes a material comprising none, or a normal, low, or high level of one of more of the marker (or biomarker) expression products of one or more the markers (or biomarkers) of the invention, such that the control or reference standard may serve as a comparator against which a sample can be compared.

The “level” of one or more target molecule means the absolute or relative amount or concentration of the molecule in the sample.

“Measuring” or “measurement,” or alternatively “detecting” or “detection,” means assessing the presence, absence, quantity or amount (which can be an effective amount) of either a given substance within a sample, including the derivation of qualitative or quantitative concentration levels of such substances.

A “nucleic acid” refers to a polynucleotide and includes poly-ribonucleotides and poly-deoxyribonucleotides. Nucleic acids according to the present invention may include any polymer or oligomer of pyrimidine and purine bases, e.g., cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide or ribonucleotide component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, at least 8, at least 15 or at least 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this disclosure. It will be understood that when a nucleotide sequence is represented herein by a DNA sequence (e.g., A, T, G, and C), this also includes the corresponding RNA sequence (e.g., A, U, G, C) in which “U” replaces “T”.

As used herein, a “polypeptide of interest” may be any polypeptide for which said polypeptide's genomic binding regions are sought. It is envisioned that a polypeptide of the present invention may include full length proteins and protein fragments. While the methods of the present invention may be utilized not only to determine at least one region of a genome at which a polypeptide of interest binds, they may also be utilized to determine if a polypeptide binds to a genome at all. The polypeptide of interest may selected from the group consisting of a transcription factor, a polymerase, a nuclease, and a histone.

“Primer” as used herein refers to a single-stranded oligonucleotide or a single-stranded polynucleotide that is extended on its 3′ end by covalent addition of nucleotide monomers during amplification. Nucleic acid amplification often is based on nucleic acid synthesis by a nucleic acid polymerase. Many such polymerases require the presence of a primer that can be extended to initiate such nucleic acid synthesis.

As used herein, “sample” or “test sample,” may refer to any source used to obtain nucleic acids for examination using the compositions and methods of the invention. A test sample is typically anything suspected of containing a target particle.

As used herein, “fluid sample” or “sample fluid” means a fluid source of analytes. Fluid samples can include blood, saliva, tears, sweat, interstitial fluid, plant biofluids, river water, fluids used in chemical processing plants, or other possible sample fluids.

“Standard control value” as used herein refers to a predetermined amount of a molecule that is detectable in a sample. The standard control value is suitable for the use of a method of the present invention, for comparing the amount of a target molecule of interest that is present in a test sample. An established sample serving as a standard control provides an average or expected amount of the target of interest in a sample, and may be used to determine a background level of detection for use as a comparator control. A standard control value may vary depending on the target molecule of interest and the nature of the sample (e.g., purified sample or patient sample).

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

### DESCRIPTION

Devices that enable detection of a target particle, nucleic acid molecule or antigen in small samples are needed for analytical and diagnostic applications within multiple fields including medicine, agriculture, environmental protection, and food processing or regulation. For example, rapid detection of pathogens or antibiotic-resistant bacteria in a small patient sample would help clinicians effectively diagnose a patient and select an effective therapeutic regimen.

The invention provides a microfluidic concentrator chip that can be used in combination with an assay for detection of a target particle or antigen to increase the local concentration of the target particle or antigen within an ion concentration polarization (ICP) region, allowing for sensitive detection of the target particle or antigen. In some embodiments, the concentrator chip of the invention allows for detection of a target molecule that exists in small quantities in a test sample. For example, in some embodiments, the concentrator chip of the invention allows for detection of a low-copy-number target nucleic acid molecule within a sample without a prior amplification step. For example, in some embodiments, the invention provides systems and methods for detecting a nucleic acid molecule using the microfluidic concentrator chip in combination with a one-step CRISPR-based detection assay.

In some embodiments, the invention provides a one-step CRISPR detection system comprising: a microfluidic concentrator chip which concentrates a target nucleic acid molecule within an ion concentration polarization (ICP) region, a Cas effector protein and one or more guide RNAs designed to bind to the target molecule. In some embodiments, the system can be used to detect a viral nucleic acid molecule in a sample (e.g., a viral RNA molecule).

### Microfluidic Concentrator Chip Device

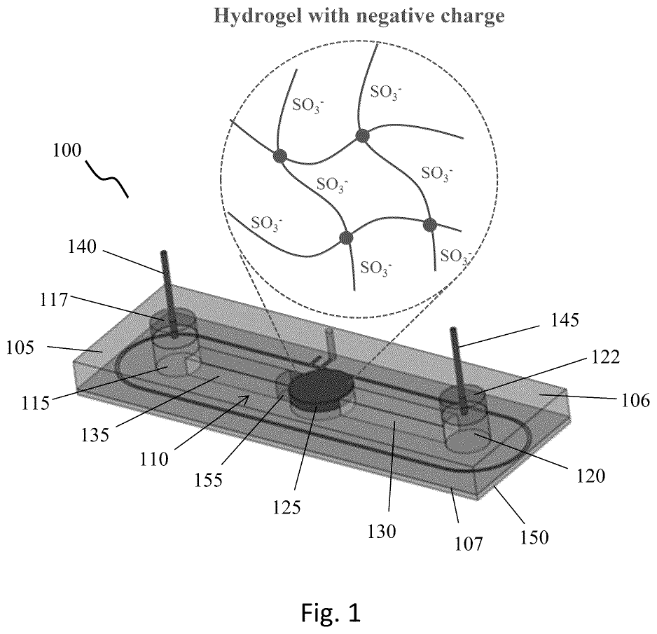

Aspects of the present invention relate to a microfluidic concentrator chip device used in some examples for the detection of pathogen, infection and/or disease in a sample. The disclosed microfluidic concentrator chip device uses a channel with a hydrogel membrane, and electrodes positioned within the channel to enable ion concentration polarization (ICP). Now referring to FIG. 1, shown is a perspective view of an exemplary microfluidic concentrator chip device 100 comprising microfluidic chip 105 comprising a flat body having a top surface 106 and a bottom surface 107. In some embodiments, microfluidic chip 105 comprises at least one channel 110 extending for a length through microfluidic chip 105, the channel having a bottom surface, a top surface, sidewalls and at least two ends. In some embodiments, channel 110 comprises a first end 115 in fluid communication with and extending to a second end 120. In some embodiments, microfluidic chip 105 further comprises two openings extending through top surface 106 in fluid communication with channel 110 and positioned diametrically opposed along channel 110. In some embodiments, the first opening comprises an inlet 117 forming an opening in top surface 106 of microfluidic chip 105 in fluid communication with first end 115, and enabling access to channel 110 from the exterior of the device. In some embodiments, a second opening comprises outlet 122 forming an opening in top surface 106 of microfluidic chip 105 in fluid communication with second end 120, and similarly for enabling access to channel 110. In some embodiments, the bottom surface of channel 110 is formed by fixedly and removably attaching a glass substrate 150 to bottom surface 107 of microfluidic chip 105.

In some embodiments, device 100 further comprises a hydrogel membrane 125 positioned within channel 110. In some embodiments, hydrogel membrane 125 is positioned in channel 110 equidistant between first end 115, and second end 120. In some embodiments, hydrogel membrane 125 is positioned in a central location or an offset location between first end 115 and second end 120. Although an example is provided wherein hydrogel membrane 125 is positioned centrally within channel 110, it should be understood that hydrogel membrane 125 is not limited to any particular location and/or position along the length of channel 110. In some embodiments, hydrogel membrane 125 has a circular pattern and/or shape. However, it should be understood that hydrogel membrane 125 is not limited to any particular shape or pattern.

Device 100 further comprises a buffer solution 130 contained within channel 110. In some embodiments, a sample 135 is placed through inlet 117, into first end 115 of channel 110, wherein sample 135 is mobilized along the length of channel 110 towards membrane 125 in buffer solution 130 to a region of interest (ROI) 155. In some embodiments, device 100 further comprises at least two electrodes, wherein a first electrode is a cathode 140 and a second electrode is an anode 145. In some embodiments, cathode 140 passes through inlet 117 and is positioned in first end 115, such that inlet 117 and/or first end 115 receives at least a portion of cathode 140, and cathode 140 is in fluid communication with buffer solution 130. In some embodiments, anode 145 passes through outlet 122 and is positioned within second end 120, such that outlet 122 and/or second end 120 receives at least a portion of anode 145, and anode 145 is in fluid communication with buffer solution 130. In some embodiments, device 100 further comprises a power supply electrically connected to anode 145 and cathode 140 and configured to apply a voltage across channel 110 and/or buffer solution 130. In some embodiments, the voltage is a direct current (DC) voltage. In some embodiments, the power supply comprises a battery. In some embodiments, the power supply comprises an AC/DC adapter. In some embodiments, the power supply may be any direct current power supply, or any power supply known in the art.

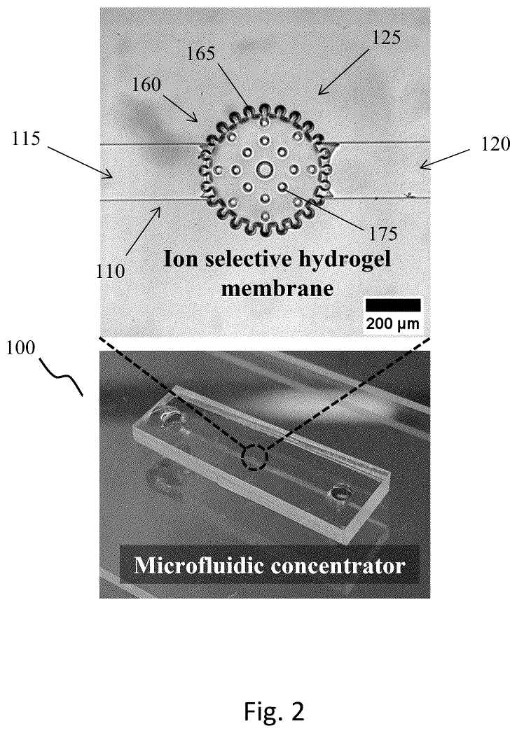

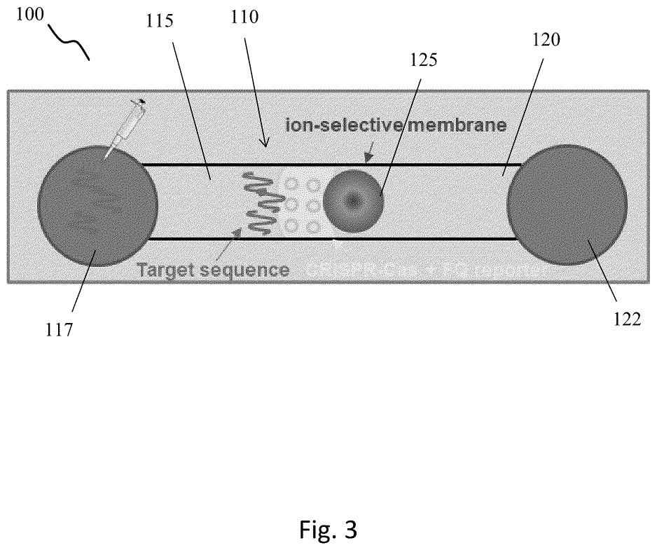

Now referring to FIG. 2, shown is a perspective view of an exemplary device 100 showing an enlarged top down view of channel 110 and hydrogel membrane 125. In some embodiments, channel 110 further comprises a cavity 160 having a bottom surface, a top surface, and sidewalls extending upwards from the bottom surface to the top surface. In some embodiments, cavity 160 is positioned along the length of channel 110 in fluid communication with channel 110, first end 115 and second end 120. In some embodiments, both membrane 125 and cavity 160 are circular in shape. In some embodiments, hydrogel membrane 125 is positioned within cavity 160. For example, in some embodiments, hydrogel membrane 125 is positioned near and/or in contact with the bottom surface of channel 110 and/or cavity 160. In some embodiments, membrane 125 is positioned near and/or in contact with the top surface of channel 110 and/or the top surface of cavity 160. Further, in some embodiments, membrane 125 may extend upwards from the bottom surface of channel 110 and/or the bottom surface of cavity 160 wherein at least a portion of membrane 125 contacts the top surface of channel 110 and/or the top surface of cavity 160.

In some embodiments, cavity 160 comprises a series of peripheral cutouts 165 arranged circumferentially around cavity 160 and forming cutout regions in the sidewalls of cavity 160. In some embodiments, peripheral cutouts 165 are arranged radially surrounding cavity 160, wherein the cutouts extend upwards from the bottom surface to the top surface of cavity 160. In some embodiments, cavity 160 further comprises one or more pillars 175 comprising cylindrical posts extending downward from the top surface of cavity 160 to the bottom surface of cavity 160. In some embodiments, pillars 175 extend downward into cavity 160 and do not make contact with the bottom surface of cavity 160. In some embodiments, pillars 175 extend upward from the bottom surface of cavity 160. In some embodiments, pillars 175 are arranged in a pattern within cavity 160. In some embodiments, pillars 175 are arranged in a circular pattern, a radial pattern, a star pattern, a concentric pattern.

In some embodiments, peripheral cutouts 165 provide a series of peripheral anchoring regions for hydrogel membrane 125, and inhibit the flow of hydrogel prepolymer solution out of cavity 160 and into channel 110 during the manufacture and/or positioning of hydrogel membrane 125. In some embodiments, peripheral cutouts 165 prevent lateral and/or rotational movement of hydrogel 125. Similarly, in some embodiments, pillars 175 serve as anchors for hydrogel 125, preventing lateral and/or rotational movement of membrane 125, and inhibit the flow of hydrogel prepolymer solution out of cavity 160 and into channel 110. In some embodiments, the combination of peripheral cutouts 165 and pillars 175 prevent lateral and/or rotational movement of membrane 125 within cavity 160 and/or channel 110.

Aspects of the present invention relate to dimensions for device 100. As contemplated herein, the heights and widths of channel 110, membrane 125, and cavity 160 enable specific electrokinetic properties of device 100. Further, the distance between first end 115 and second end 120, and the length of channel 110 enable specific electrokinetic properties of device 100. In some embodiments, channel 110 has height of about 10 μm, 15 μm, 20 μm, 25 μm, 30 μm, 35 μm, 40μ, 45μ, 50μ, 55μ, 60μ, 65 μm, 70μ, 75μ, 80μ, 85μ, 90μ, 95 μm, 100 μm, 110 μm, 120 μm, 120 μm, 130 μm, 140 μm, 150 μm, 160 μm, 170 μm, 180 μm, 190 μm, 200μ, 210 μm, 220 μm, 230 μm, 240 μm, 250μ, 260 μm, 270 μm, 280 μm, 290 μm, or 300 μm. In some embodiments, channel 110 has a length of about 200 μm, 210 μm, 220 μm, 230 μm, 240 μm, 250 μm, 260 μm, 270 μm, 280 μm, 290 μm, 300 μm, 500 μm, 750 μm, or 1000 μm. For example, in certain embodiments the height of channel 110 is 20 μm and the length is 750 μm.

In some embodiments, channel 110 has an aspect ratio (height:width) of about 1:1, 1:2, 1:3, 1:4, 1:5, 1:6, 1:7, 1:8, 1:9, or about 1:10. In some embodiments, channel 110 has width of about 100 μm, 150 μm, 200 μm, 210 μm, 220 μm, 230 μm, 250 μm, 300μ, 400 m, 450 μm, 500 μm, 550 μm, 600 μm, 650 μm, 700 μm, 750 μm, 800 μm, 850 μm, 900 μm, 950 μm, 1000 μm, 1100 μm, 1200 μm, 1300 μm, 1400 μm, 1500 μm, 1600 μm, 1700 μm, 1800 μm, 1900 μm, 2000 μm, 2100 μm, 2200 μm, 2300 μm, 2400 μm, 2500 μm, 2600 μm, 2700 μm, 2800 μm, 2900 μm, or 3000 μm. In some embodiments, channel 110 has a height of about 100 μm, 150 μm, 200 μm, 210 μm, 220 μm, 230 μm, 250 μm, 300 μm, 400 μm, 450 μm, 500 μm, 550 μm, 600 μm, 650 μm, 700 μm, 750 μm, 800 μm, 850 μm, 900 μm, 950 μm, 1000 μm, 1100 μm, 1200 μm, 1300 μm, 1400 μm, 1500 μm, 1600 μm, 1700 μm, 1800 μm, 1900 μm, 2000 μm, 2100 μm, 2200 μm, 2300 μm, 2400 μm, 2500 μm, 2600 μm, 2700 μm, 2800 μm, 2900 μm, or 3000 μm For example, in certain embodiments the width of channel 110 is 200 μm and the height is 2000 μm.

In some embodiments, the height and width of hydrogel membrane 125 is sized to fill at least a portion of channel 110 and/or cavity 160. In some embodiments, membrane 125 has a height of about 1.5 μm, 2.0 μm, 2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm, 9.5 μm, 10 μm, 10.5 μm, 11 μm, 11.5 μm, 12μ, 12.5μ, 13 μm, 13.5 μm, 14 μm, 14.5 μm, 15μ, 15.5 μm, 16 μm, 16.5 μm, 17 μm, 17.5 μm, 18 μm, 18.5 μm, 19 μm, 19.5 μm, 20 μm, 22.5 μm, 25 μm, 27.5 μm, 30 μm, 32.5 μm, 35 μm, 37.5 μm, 40 μm, 45 μm, 50 μm, 55 μm, 60 μm, 65 μm, 70 μm, 75 μm, 80 μm, 85 μm, 90 μm, or 100 μm. For example, in certain embodiments the height of membrane 125 is 6.0 μm. In some embodiments, the height of membrane 125 is a determined by a defined ratio of height of membrane 125 to the height of channel 110. In some embodiments, the ratio of the height of membrane 125 to the height of channel 110 is about 1:6, 1:5, 1:4, 1:3, 1:2, 2:3, 3:5, 3:4, or about 5:6. For example, in some embodiments the height of membrane 125 is approximately 20%-80% of the height of channel 110. For example, in some embodiments the height of hydrogel membrane 125 is approximately 25%-40% of the height of channel 110. In some embodiments the height of hydrogel membrane 125 is approximately 30% of the height of channel 110. In some embodiments, the height of hydrogel membrane 125 is about 6 μm and the height of channel 110 is about 20 μm.

In some embodiments, the width of hydrogel membrane 125 is about twice the width of channel 110. In some embodiments, hydrogel membrane 125 has a width of approximately 200 μm, 300 μm, 400 μm, 420 μm, 440 μm, 460 μm, 500 μm, 600 μm, 800 μm, 900 μm, 1000 μm, 1100μ, 1200μ, 1300 μm, 1400μ, 150 μm, 1600 μm, 1700 μm, 1800 μm, 1900 μm, 2000 μm, 2200 μm, 2400 μm, 2600 μm, 2800 μm, 3000 μm, 3200 μm, 3400 μm, 3600 μm, 3800 μm, 4000 μm, 4200 μm, 4400 μm, 4600 μm, 4800 μm, 5000 μm, 5200 μm, 5400 μm, 5600 μm, 5800 μm, or 6000 μm.

In some embodiments, a distance between either channel 110 or cavity 160 and hydrogel membrane 125 allows buffer solution 130 to flow in between membrane 125 and either the top and/or bottom surface of channel 110 or cavity 160. In some embodiments, distance is about 20-80% of the height of channel 110 and/or cavity 160. In some embodiments, distance is about 60-75% of the height of channel 110 and/or cavity 160. In some embodiments, the distance is about 2μ, 3 μm, 4μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 22 μm, 24 μm, 26 μm or about 28 μm. For example, in some embodiments, distance is about 14 μm.

Inlet 117 and outlet 122 form, in some examples, round openings in microfluidic chip 105 to access first end 115 and second end 120, respectively. In some embodiments, the diameter of inlet 117 and outlet 122 are the same. In some embodiments, the diameter of inlet 117 and outlet 122 are different. In some embodiments, the diameters may be about 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm or about 2.5 mm. For example, in some embodiments, the size of inlet 117 and outlet 122 are the same and have a diameter of 1.5 mm. In some embodiments, inlet 117 and outlet 122 are configured to receive a stopper or plug to seal channel 110 from the environment and/or exterior of device 100.

Aspects of the present invention relate to inducing electrokinetic characteristics of device 100 and/or providing an electrical gradient across channel 110 and/or cavity 160. In some embodiments, a direct current (DC) voltage is applied across cathode 140 and anode 145 to induce selective transport of cations via ion concentration polarization (ICP). In some embodiments, a voltage is applied of about 0.1V, 0.5V, 1V, 2V, 3V, 4V, 5V, 6V, 7V, 8V, 9V, 10V, 15V, 20V, 25V, 30V, 35V, 40V, 45V, 50V, 55V, 60V, 65V, 70V, 75V or about 80V. For example, in some embodiments, a voltage of 60V is applied across cathode 140 and anode 145 and channel 110 and/or cavity 160. In some embodiments, the voltage depends on the amount of current required to generate bubbles around the electrodes, and may be modulated to increase or reduce the bubbling effect and/or the resulting transport of cations. In some embodiments, the electrical current generated is dependent on the electrical resistance of channel 110 that is dependent on the length, height and width of the channel 110.

Aspects of the present invention relate to one or more materials for device 100. In some embodiments, device 100 comprises Polydimethylsiloxane (PDMS). In some embodiments, device 100 comprises any of polystyrene, polymethyl methacrylate (PMMA), poly(lactic-co-glycolic acid) (PLGA), elastomer, silicon, glass, cyclic olefin copolymer (COC), polyester or other thermoplastic polymers. Although example materials are provided, device 100 may comprise any material that would be known by someone with an ordinary level of skill in the art.

Aspects of the present invention relate to hydrogel materials for device 100 and/or hydrogel membrane 125. In some embodiments, hydrogel membrane 125 comprises any hydrogel material that would be known by one of ordinary level of skill in the art. In some embodiments, hydrogel membrane 125 comprises a hydrogel material having a highly negatively charged sulfonate group that allows selective transport of cations through channel 110 and/or cavity 160 from first end 115 to second end 120. In some embodiments, hydrogel membrane 125 comprises a hydrogel comprising 2-Acrylamido-2-methylpropane sulfonic acid as a monomer, N,N′-Methylenebisacrylamide as a crosslinker, (2-Hydroxy-1-(4-(2-hydroxyethoxy)phenyl)-2-methylpropan-1-one) as a photo initiator in dimethyl sulfoxide (DMSO). In some embodiments, any hydrogel of device 100 may be set and/or polymerized with a UV light. In some embodiments, hydrogel membrane 125 comprises 2-Acrylamido-2-methylpropane sulfonic acid as a monomer and N,N′-Methylenebisacrylamide as a crosslinker that has undergone photoinduced polymerization. Other exemplary hydrogel materials that may be used include, but are not limited to, Nafion and PEDOT:PSS (poly(3,4-ethylenedioxythiophene) polystyrene sulfonate).

In some embodiments, channel 110 further comprises one or more embedded or functionalized reagents for detecting a target in a sample. In some embodiments, the one or more embedded or functionalized agents are positioned within channel 110, cavity 160 and/or hydrogel membrane 125. Exemplary reagents include, but are not limited to, oligonucleotide probes, antibodies, antibody fragments, guideRNAs, CRISPR-Cas polypeptides, and the like. In some embodiments, device 100 may comprise one or more biosensing elements positioned within channel 110, cavity 160 and/or hydrogel 125, including but not limited to, nanowires, ring resonators, and the like.

In some embodiments, channel 110 and/or hydrogel membrane 125 comprise a buffer solution 130 as a medium to facilitate ion concentration polarization (ICP) and/or other electrochemical transport phenomena. In some embodiments, the ICP and/or other electrochemical transport phenomena occur within channel 110 and across hydrogel membrane 125. As contemplated herein, any suitable biological buffer solution may be used that would be known by one of ordinary level of skill in the art. In some embodiments, buffer solution 130 comprises NEBuffer (New England Biolabs). In some embodiments, buffer solution 130 comprises any of 50 mM NaCl, 10 mM Tris-HCl, 10 mM MgCl₂, 100 g/ml Recombinant Albumin (pH 7.9@25° C.) and combinations thereof. In some embodiments, buffer solution 130 comprises reagents for detecting a target in a sample. Exemplary reagents include, but are not limited to, oligonucleotide probes, antibodies, antibody fragments, guideRNAs, CRISPR-Cas polypeptides, and the like. In some embodiments, buffer solution 130 comprises reagents required for CRISPR/Cas enzyme-mediated diagnostics, such as SHERLOCK and DETECTR. In some embodiments, buffer solution 130 comprises at least one ribonuclease (RNAse) inhibitor. In some embodiments, the RNase inhibitor (RI) is a protein binding to ribonucleases (RNases) that effectively inhibits the enzymatic activity of the RNases, thus preserving the integrity and stability of RNA samples. In some embodiments, the RNase inhibitor comprises a broad-spectrum of inhibition, and should be effective against following RNases such as RNase A, B, C, 1, and T1 which are the most common and troublesome RNases. For example, in some embodiments, buffer solution 130 comprises any of RNase Inhibitor of Murine origin (NEB), RNaseOUT™ Recombinant Ribonuclease Inhibitor (ThermoFisher), and SUPERase•In™ RNase InhibitorRNase inhibitor (ThermoFisher), and any combinations thereof.

In some embodiments, device 100 may be configured for multiplexing. In some embodiments, microfluidic chip 105 comprises one or more channels 110, wherein the channels are arranged parallel on microfluidic chip 105. In some embodiments, device 100 comprises one or more microfluidic chips 105. In some embodiments, the one or more microfluidic chips 105 comprise one or more channels 110 for assay multiplexing. For example, in some embodiments, device 100 comprises two chips 105 comprising an array of channels 110. In some embodiments, the one or more channels 110 allow for multiplexed detection of a plurality of targets, for example each channel being configured for a specific target. In some embodiments, the one or more channels 110 allows for multiplexed detection of a specific target across a plurality of samples, for example each channel being specific sample. In some embodiments, each channel 110 comprises one or more hydrogel membranes 125. In some embodiments, one or more channels 110 share a single hydrogel membrane 125, wherein the hydrogel membrane 125 is positioned across the one or more channels 110. For example, in some embodiments, a hydrogel membrane 125 may be positioned in the center of two or more channels 110 that are arranged in a cruciform pattern on microfluidic chip 105.

In some embodiments, device 100 as described can be coupled with other devices, systems and assays to incorporate the results of the device with other technologies. For example, in some embodiments device 100 can be coupled to microarray plates, nanowires, ring resonators, or any other applicable device, system, or assay used for biosensing or diagnostic detection of a desired target analyte.

### Fabrication Steps of the Microfluidic Concentrator Chip

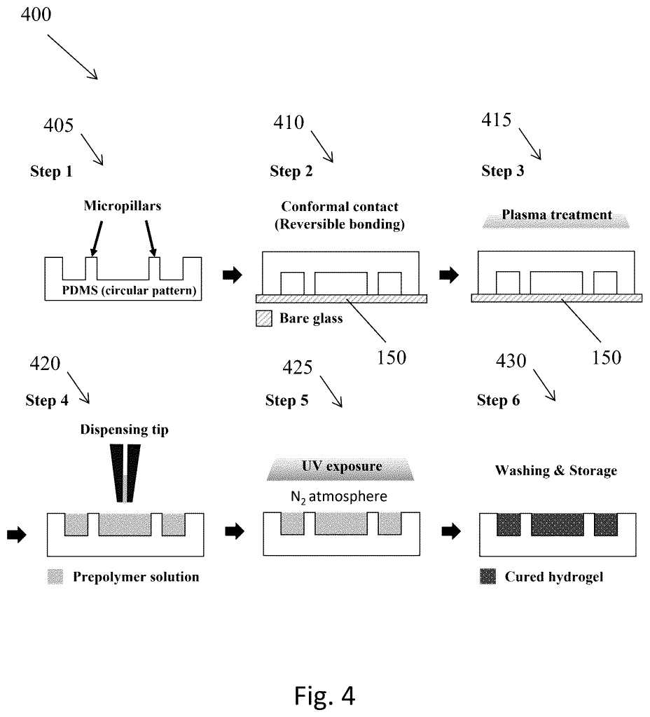

Aspects of the present invention relate a fabrication method for device 100. Now referring to FIG. 4, depicted is an exemplary process 400 comprising the steps involved for the fabrication and/or preparation of device 100. In a first step, a single-channel PDMS microfluidic chip 105 with an array of micropillars 175 is surface cleaned with adhesive tape and IPA thoroughly (405). Next, device 100 is placed on top of a glass substrate 150 and fixedly and removably sealed against a glass substrate 150 (410). Next, device 100 is then plasma treated for 1 min to induce hydrophilicity inside channel 110 (415). Next, the glass substrate 150 is removed from device 100 and microfluidic chip 105 is opened. After opening microfluidic chip 105, it is then placed on glass substrate 150 with the channel side facing up. Next, a prepolymer solution composed of 2-Acrylamido-2-methylpropane sulfonic acid as a monomer, N,N′-Methylenebisacrylamide as a crosslinker, (2-Hydroxy-1-(4-(2-hydroxyethoxy)phenyl)-2-methylpropan-1-one) as a photo initiator in dimethyl sulfoxide (DMSO) is pipetted on a circular pattern at the center of channel 110 (420). The loaded prepolymer solution on the microfluidic chip 105 is then exposed with UV light (365 nm) for 6 min. within an N2 gas atmosphere, forming a crosslinked cation-selective hydrogel membrane on microfluidic chip 105 (425). Lastly, the hydrogel layer is washed by deionized water for several times and stored at room temperature before use (430).

### Method of Use

In some embodiments, the present invention provides exemplary methods of use for a microfluidic concentrator chip (e.g. device 100 of the present invention). In some embodiments, the methods relate to concentrating a target analyte in a sample. In some embodiments, exemplary target analytes include, but are not limited to pathogens, soluble antigens, nucleic acids, proteins small molecules, toxins, chemicals, plant pathogens, blood borne pathogens, bacteria, viruses and the like. In some embodiments, device 100 allows for the target analyte to be detected while in the ion concentration polarization (ICP) region (channel 110) of microfluidic chip 105. In some embodiments, the concentrated target analyte can be released from channel 110 and microfluidic chip 105 (e.g. following disruption of the ICP region by turning off the voltage or applying higher fluidic pressure for instance).

In some embodiments, device 100 can be incorporated into a system for evaluating the level or amount of a target analyte in a sample. For example, in some embodiments, outlet 120 of device 100 is connected to a detector, a sensor, or a sample collection device. Exemplary detectors include, but are not limited to, fluorescence detectors, electrochemical detectors, a mass spectrometer, including sample deposition onto a matrix assisted laser desorption/ionization (MALDI) plate, an optical microring resonator, a surface plasmon resonance (SPR) detector and conductivity detectors.

Aspects of the present invention provide an exemplary method of use for concentrating a target analyte using device 100. In some embodiments the method for concentrating and/or detecting a target analyte comprises flowing a test sample through one or more channels on device 100, and applying a direct current (DC) voltage across the one or more channels to generate an ion concentration polarization (ICP) region. In some embodiments, the method further comprises releasing the concentrated analyte sample from device 100.

In some embodiments, the method for concentrating and/or detecting a target analyte in a sample comprises mixing a test sample with a detectable molecule and flowing the sample through the one or more channel on device 100, and applying a direct current (DC) voltage across the one or more channels to generate an ion concentration polarization (ICP) region and detecting one or more molecules in the ICP region of the one or more channels.

In some embodiments, the method for concentrating and/or detecting a target analyte in a sample comprises flowing a test sample through one or more channels on device 100, applying a direct current (DC) voltage across the one or more channels to generate an ion concentration polarization (ICP) region to concentrate the target analyte, stopping the DC voltage, and/or applying higher fluidic pressure to disrupt the ICP region to release the concentrated analyte sample, and detecting the presence of the target analyte in the concentrated sample.

In some embodiments, the method for concentrating and/or detecting a target analyte in a sample comprises heating a sample to a set temperature for a period of time. In some embodiments, the sample is heated to about 25° C., 30° C., 35° C., 40° C., 45° C., 50° C., 55° C., 60° C. or about 65° C. In some embodiments, the sample is heated for about 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, or about 20 min. For example, in some embodiments, the sample is heated to about 37° C. for about 10 min. In some embodiments, the temperature and period of time may be modified depending on the design and conditions of the experiment for optimal activity of enzymes.

In some embodiments, device 100 can be used for performing one or more on-chip assays. In some embodiments, device 100 can be used for concentrating a sample for performing one or more down-stream assays. Exemplary assays include, but are not limited to, an immunoassay, a microarray, a DNA hybridization assay, a morpholino assay, an aptamer assay, a peptide nucleic acid (PNA) assay, and a target analyte detection assay.

In some embodiments, device 100 can be used to concentrate target molecules for detection using an immunoassay. Suitable immunologic assay methods include, but are not limited to, immunoprecipitation, particle immunoassay, immunonephelometry, radioimmunoassay (RIA), enzyme immunoassay (EIA) including enzyme-linked immunosorbent assay (ELISA), sandwich, direct, indirect, or competitive ELISA assays, enzyme-linked immunospot assays (ELISPOT), multiplex ELISA array, fluorescent immunoassay (FIA), chemiluminescent immunoassay, flow cytometry assays, immunohistochemistry, Western blot, integrated blood barcode chip and protein-chip assays.

In some embodiments, a region of channel 110 of device 100 which will become the ICP region when DC voltage is applied is functionalized or embedded with one or more capture agents for the capture and subsequent detection of a target analyte, nucleic acid molecule or antigen in the ICP region of the channel. Exemplary capture agents that can be used to functionalize the ICP region of the channel of device 100 include, but are not limited to oligonucleotides, peptides, antibodies, and other binding molecules.

In some embodiments, detection of an analyte, nucleic acid molecule or antigen in the ICP region of device 100 or in a concentrated analyte sample is based on the affinity between the target analyte (e.g., antigen, antibody or nucleic acid molecule) and a detector molecule. As contemplated herein, detection method may be performed as any type of affinity binding assay or immunoassay, as would be understood by those skilled in the art. In some embodiments, a detector molecule binds directly or indirectly to a target analyte (e.g., a nucleic acid molecule or antigen) in the ICP region of device 100 allowing for direct detection of the target analyte in the ICP region. In some embodiments, a detector molecule becomes activated in the presence of a target analyte (e.g., a nucleic acid molecule or antigen) in the ICP region of device 100 allowing for detection of the target analyte in the ICP region. In some embodiments, a detector molecule is applied to channel 110 of device 100 following binding of a target analyte to a capture molecule embedded or functionalized in the ICP region of device 100. In some embodiments, a concentrated analyte sample is contacted with a detector molecule following release from device 100.

In some embodiments, the detectable molecule can be a nucleic acid sequence, an amino acid sequence, an antibody, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The antibody can be a monoclonal antibody or antibody fragment (e.g., an scFv antibody fragment.) The polysaccharide can be a nucleic acid encoded polysaccharide. In some embodiments, the detectable molecule is a fluorescent probe that is quenched in the absence of the target analyte, but is activated when the target analyte is present. Exemplary fluorescent quencher probes are labeled with two different dyes, e.g., a fluorophore and a quencher. When the two dyes are in close proximity, as is the case in an intact oligonucleotide probe, one of the dyes (e.g., TAMRA [N,N,N′,N′-tetramethyl-6carboxyrhodamine]) can act as a quencher for a second fluorescent dye (e.g., FAM [5-carboxyfluorescein]) by absorbing at the FAM emission spectra. In some embodiments, binding of a target molecule to the fluorescent quencher probe separates the two dyes to allow fluorescence to be detected. In some embodiments, the fluorescent quencher probe is cleaved in the presence of a target analyte which separates the two dyes to allow fluorescence to be detected.

In some embodiments, the invention provides exemplary methods for detecting a target nucleic acid molecule or antigen from a biological sample, such as a bodily fluid sample or a tissue sample. It should be appreciated that any biological sample may be used, or any tissue type, provided such samples carry the targeted nucleic acid molecule or antigen to be analyzed. Exemplary fluid samples that can be analyzed using the methods of the invention include, but are not limited to, urine, saliva, blood, serum, plasma, amniotic fluid, mucus, or tears. In some embodiments, the sample will be a “clinical sample” which is a sample derived from a patient. In some embodiments, the biological sample is a blood sample. In some embodiments, the biological sample is a serum sample or a plasma sample, derived from a blood sample of the subject.

### Viral Nucleic Acid Molecules or Antigens

In some embodiments, the target nucleic acid molecule or antigen comprises a viral nucleic acid molecule or antigen, or fragment thereof, or variant thereof. In some embodiments, the viral antigen is from a virus from one of the following families: Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, or Togaviridae. In some embodiments, the viral antigen is from papilloma viruses, for example, human papilloma virus (HPV), human immunodeficiency virus (HIV), polio virus, hepatitis B virus, hepatitis C virus, smallpox virus (Variola major and minor), vaccinia virus, influenza virus, rhinoviruses, dengue fever virus, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, chikungunya virus, lassa virus, arenavirus, severe acute respiratory syndrome (SARS) virus, severe acute respiratory syndrome coronavirus 2 (SARS-COV-2) or a cancer causing virus.

### Parasite Nucleic Acid Molecules or Antigens

In some embodiments, the target nucleic acid molecule or antigen comprises a parasite nucleic acid molecule or antigen or fragment or variant thereof. In some embodiments, the parasite is a protozoa, helminth, or ectoparasite. In certain embodiments, the helminth (i.e., worm) is a flatworm (e.g., flukes and tapeworms), a thorny-headed worm, or a round worm (e.g., pinworms). In certain embodiments, the ectoparasite is lice, fleas, ticks, and mites.

In some embodiments, the parasite is any parasite causing the following diseases: Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis.

In certain embodiments, the parasite is Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus-lung fluke, Pinworm, Plasmodium falciparum, Schistosoma, Strongyloides stercoralis, Mite, Tapeworm, Toxoplasma gondii, Trypanosoma, Whipworm, or Wuchereria bancrofti.

### Bacterial Nucleic Acid Molecules or Antigens

In some embodiments, the target nucleic acid molecule or antigen comprises a bacterial nucleic acid molecule or antigen or fragment or variant thereof. In some embodiments, the bacterium is from any one of the following phyla: Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and Verrucomicrobia.

In some embodiments, the bacterium is a gram positive bacterium or a gram negative bacterium. In some embodiments, the bacterium is an aerobic bacterium or an anaerobic bacterium. In some embodiments, the bacterium is an autotrophic bacterium or a heterotrophic bacterium. In some embodiments, the bacterium is a mesophile, a neutrophile, an extremophile, an acidophile, an alkaliphile, a thermophile, psychrophile, halophile, or an osmophile.

In some embodiments, the bacterium is an anthrax bacterium, an antibiotic resistant bacterium, a disease causing bacterium, a food poisoning bacterium, an infectious bacterium, Salmonella bacterium, Staphylococcus bacterium, Streptococcus bacterium, or tetanus bacterium. In some embodiments, bacterium is a mycobacteria, Clostridium tetani, Yersinia pestis, Bacillus anthracis, methicillin-resistant Staphylococcus aureus (MRSA), or Clostridium difficile.

### Fungal Nucleic Acid Molecules or Antigens

In some embodiments, the target nucleic acid molecule or antigen comprises a fungal antigen or fragment or variant thereof. In some embodiments, the fungus is Aspergillus species, Blastomyces dermatitidis, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium.

### Tumor Nucleic Acid Molecules or Antigens

In some embodiments, the target nucleic acid molecule or antigen comprises a tumor nucleic acid molecule or antigen, including for example a tumor-associated antigen or a tumor-specific antigen. In the context of the present invention, “tumor antigen” or “hyperporoliferative disorder antigen” or “antigen associated with a hyperproliferative disorder” refer to antigens that are common to specific hyperproliferative disorders. In some embodiments, the hyperproliferative disorder antigens of the present invention are derived from cancers including, but not limited to, primary or metastatic melanoma, mesothelioma, thymoma, lymphoma, sarcoma, lung cancer, liver cancer, non-Hodgkin's lymphoma, Hodgkins lymphoma, leukemias, uterine cancer, cervical cancer, bladder cancer, kidney cancer and adenocarcinomas such as breast cancer, prostate cancer, ovarian cancer, pancreatic cancer, and the like.

Tumor antigens are proteins that are produced by tumor cells that elicit an immune response, particularly T-cell mediated immune responses. In some embodiments, the tumor antigen of the present invention comprises one or more antigenic cancer epitopes immunogenically recognized by tumor infiltrating lymphocytes (TIL) derived from a cancer tumor of a mammal. The selection of the antigen will depend on the particular type of cancer to be treated or prevented by way of the composition of the invention.

Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

### One-Step CRISPR Based Detection

In some embodiments, the invention provides a one-step CRISPR based detection assay for detection of a target nucleic acid molecule on the microfluidic concentrator chip. CRISPR methodologies employ a CRISPR-associated (Cas) protein, that complexes with small RNAs as guides (gRNAs). Cas and guide RNA (gRNA) may be synthesized by known methods. In some embodiments, a guide RNA (gRNA) targeted to a nucleic acid molecule, and a CRISPR-associated (Cas) protein form a complex to which can be used to detect binding of the gRNA to the target nucleic acid molecule.

Non-limiting examples of Cas proteins include Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2. Csm2, Csm3, Csm4, 30 Csm5, Csm6, Cmr1, Cmr3, Cmr4, Cmr5, Cmr6, Csb1, Csb2, Csb3, Csx17, Csx14, Csx10, Csx16, CsaX, Csx3, Csxl, Csx15, Csf1, Csf2, Csf3, Csf4, SpCas9, StCas9, NmCas9, SaCas9, CjCas9, CjCas9, AsCpf1, LbCpf1, FnCpf1, VRER SpCas9, VQR SpCas9, xCas9 3.7, homologs thereof, orthologs thereof, or modified versions thereof. In some embodiments, the Cas protein has DNA or RNA cleavage activity. In some embodiments, the Cas protein is a catalytically dead variant of a Cas protein that does not have DNA or RNA cleavage activity.

In some embodiments, the Cas protein is a Cas12a or Cas13a enzyme.

In some embodiments, the method comprises applying a Cas enzyme, a gRNA molecule specific for binding to a target nucleic acid molecule, a test sample and a detectable molecule (e.g., a fluorescent marker) to a channel of the microfluidic concentrator chip, applying a DC voltage across the chip and detecting a readout (e.g., fluorescence) in the ICP region of the microfluidic concentrator chip when the gRNA molecule binds to the target nucleic acid molecule.

In some embodiments, the detectable molecule is a fluorescent quencher probes that is cleaved by an activated Cas protein upon binding of the gRNA molecule to the target nucleic acid molecule.

In some embodiments the target nucleic acid molecule is a disease-associated nucleic acid molecule. For example, in some embodiments, the target nucleic acid molecule is a viral nucleic acid molecule or a bacterial nucleic acid molecule. Therefore, in some embodiments, the invention provides a method for diagnosing a bacterial or viral disease or disorder based on the detection of a bacterial or viral nucleic acid molecule in a sample from a subject.

In some embodiments, the target nucleic acid molecule is a viral SARS-COV-2 RNA molecule. Therefore, in some embodiments, the invention provides a method for diagnosing SARS-COV-2 based on the detection of a SARS-COV-2 RNA molecule in a sample from a subject.

In some embodiments, the invention further provides methods of treating a detected disease or disorder. In some embodiments, the invention relates to a method of treating or preventing a disease or disorder in a subject in need thereof, the method comprising the step of administering a therapeutic agent for the treatment of a disease or disorder associated with a detected viral nucleic acid molecule or a bacterial nucleic acid molecule. In some embodiments, the detected viral nucleic acid molecule is a SARS-COV-2 RNA molecule and the method comprises administering a therapeutic agent for the treatment or prevention of COVID-19.

In some embodiments, the therapeutic agent is an antiviral agent. In some embodiments, the therapeutic is an antibiotic agent. In some embodiments, the therapeutic agent is a SARS-CoV-2 vaccine. In some embodiments, the therapeutic agent is a small-molecule drug or biologic.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the system and method of the present invention. The following working examples therefore, specifically point out the exemplary embodiments of the present invention, and are not to be construed as limiting in any way the remainder of the disclosure.

### Example 1: An Amplification-Free Detection of Nucleic Acids by CRISPR/Cas in an Electrokinetic Microfluidic Concentrator Chip

There have been various amplification-free CRISRP-Cas detection methods published so far (Zhang et. al., 2021, Front Microbiol, 12). However, the majority of the devices requires a special immiscible fluid for generating droplets and a pressure pump (Tian et al., 2021, Acs Nano, 15:1167-1178), microwells with a diameter of 2.5 μm (Shinoda et al., 2021, Commun Biol, 4) or adding another CRISPR nuclease Csm6 (Liu et al., 2021, Nat Chem Biol, 17:982-988) that all add more complexity to the device design, fabrication and reagents. Detailed herein is a simple device design consisting of a single microfluidic channel that can easily be fabricated using standard lithography technique and require only commercialized reagent kits. Furthermore, this detection platform is demonstrated with both Cas12a and Cas13a detection, adding versatility to the detection platform. This one step detection platform can be applicable to the detection of various viral infections including SARS-COV-2 without amplification process and could accelerate the point-of-care diagnostics.

One-Step CRISPR-Cas12a Detection of Synthetic SARS-COV-2 cDNA by Using the Microfluidic Concentrator Chip with Ion Concentration Polarization (ICP)

In general, molecular amplification processes such as Polymerase Chain Reaction (PCR), Recombinase Polymerase Amplification (RPA) and Loop-Mediated Isothermal Amplification (LAMP) are required for the detection of SARS-COV-2 due to low numbers of target RNAs in samples. However, this amplification method is labor-intensive and prolongs entire detection time.

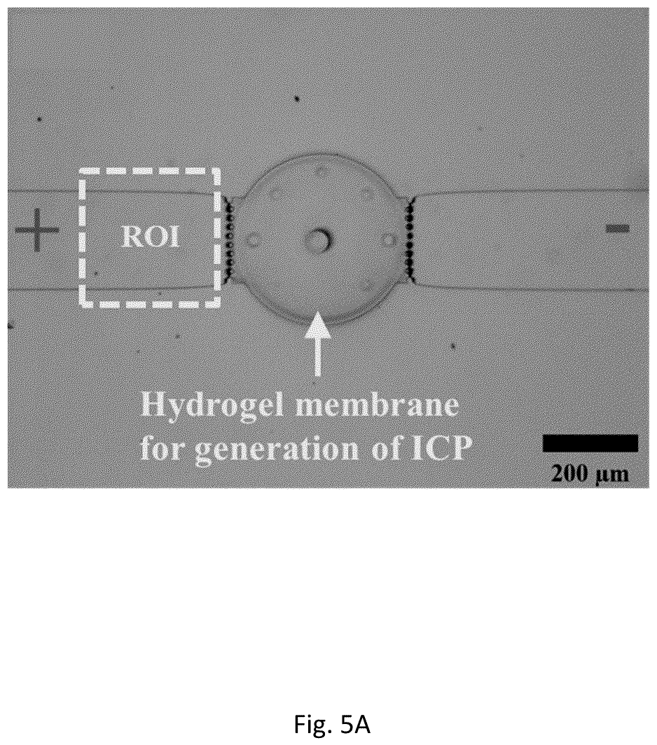

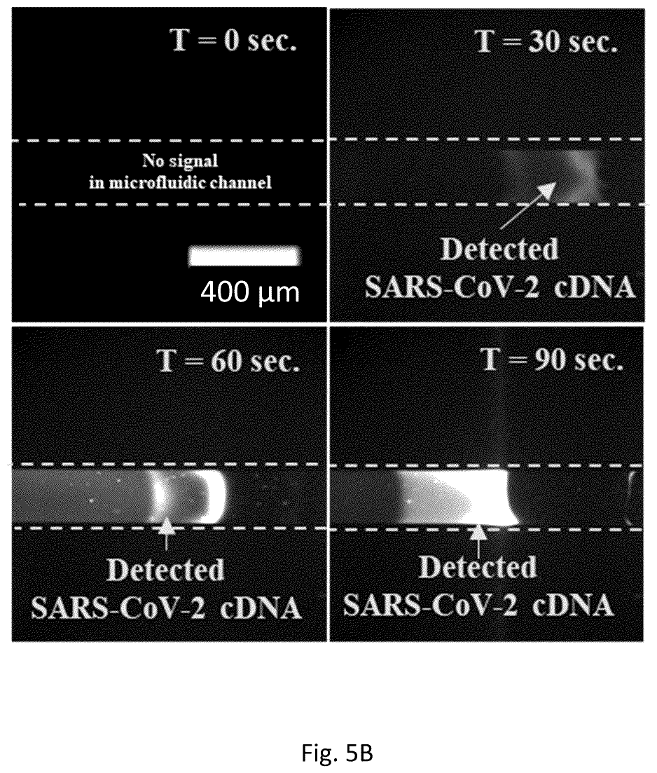

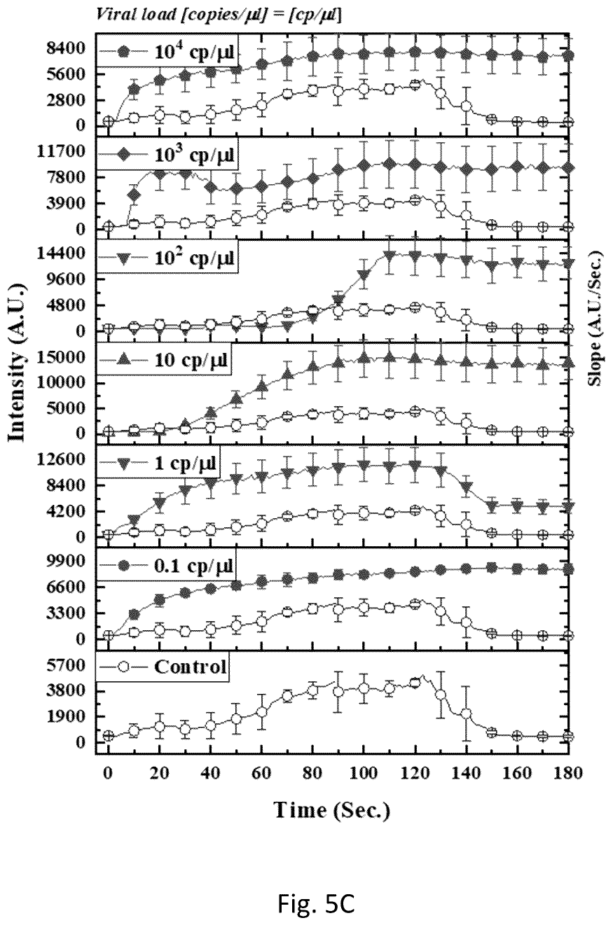

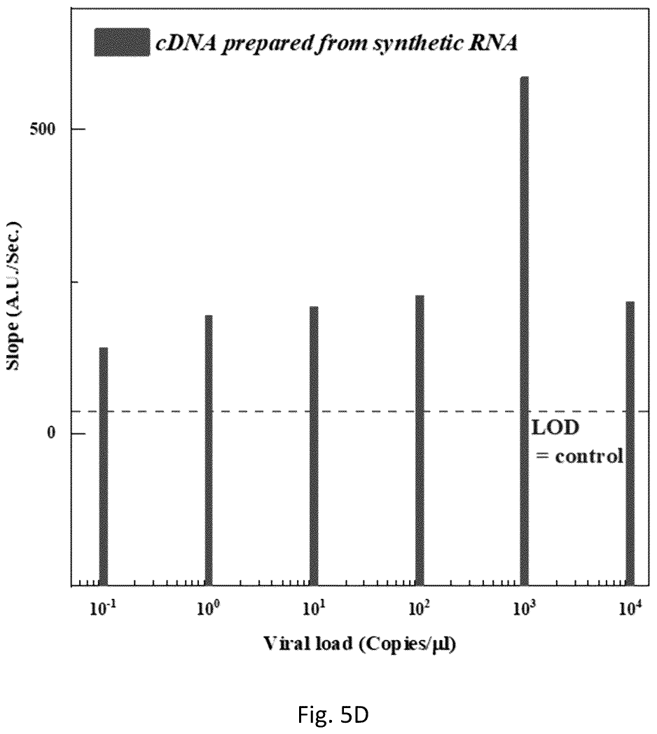

To overcome this issue, a one-step CRISPR-Cas detection method of SARS-COV-2 cDNA was developed by using an integrated microfluidic chip with ion concentration polarization (ICP). To characterize the performance of the chip, the complementary DNA (cDNA) was synthesized from the synthetic SARS-COV-2 RNA by reverse transcription. Next, the mixture composed of Cas 12a enzyme, a reaction buffer, and the target cDNA with varying copy numbers was injected and a voltage of 60 V was applied to across the microfluidic channel. When the voltage was applied across the channel, negatively charged cDNA molecules including fluorescent reporters were concentrated on the anodic side of the channel, and its fluorescence signal intensity was measured in the region of interest (ROI) (FIG. 5A).

Within 90 sec., strong fluorescence signal from the reporters was observed inside the channel due to the preconcentration by ICP, implying a successful detection of SARS-COV-2 cDNA (FIG. 5B). Notably, this detection was achieved without any molecular amplification processes such as PCR, RPA or LAMP of SARS-COV-2 cDNA, and the detection time was less than 5 minutes. Without being bound by theory, it is believed that the fast enzyme kinetics are enabled by the strong microvortices with a mixing effect, preconcentration of Cas 12a enzyme and the target cDNA in the highly localized area induced by ICP. The average intensity plot in FIG. 5C and the initial maximal slope of the CRISPR-Cas reaction calculated from the fluorescence signal intensity plot before reaching the highest intensity value in FIG. 5D demonstrated that our approach can detect from 1.0×10⁵ copies/μl down to 1 copy/μl, even in the presence of a slight background from the control samples.

The one-step CRISPR-Cas12a detection using the integrated microfluidic concentrator chip with ICP has allowed ultrafast detection of synthetic SARS-COV-2 cDNA within 5 minutes at a high sensitivity of 1 copy/μl. This approach provides a highly promising screening method to detect the presence of target nucleic acids rapidly without performing Polymerase Chain Reaction (PCR) or any other isothermal amplification methods.

One-Step CRISPR-Cas13a Detection of Synthetic SARS-COV-2 RNA by Using the Microfluidic Concentrator Chip with Ion Concentration Polarization (ICP)

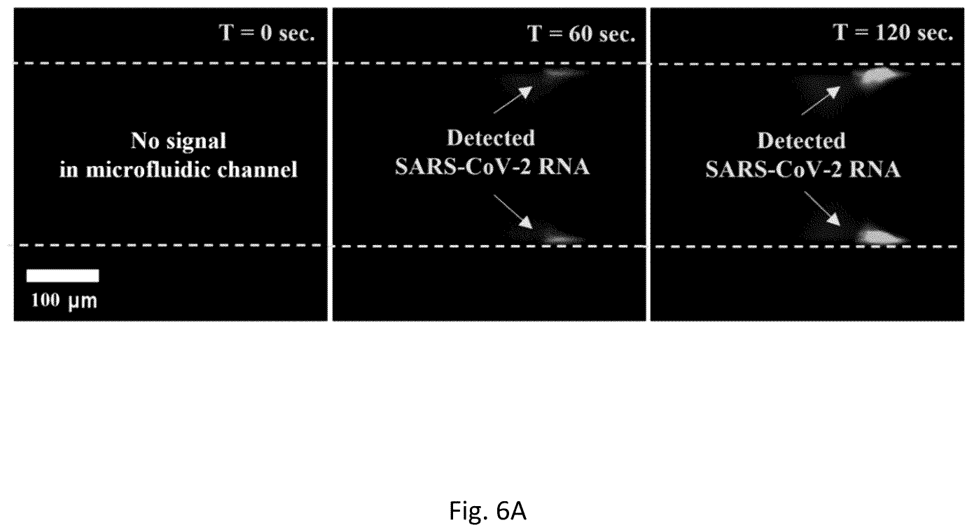

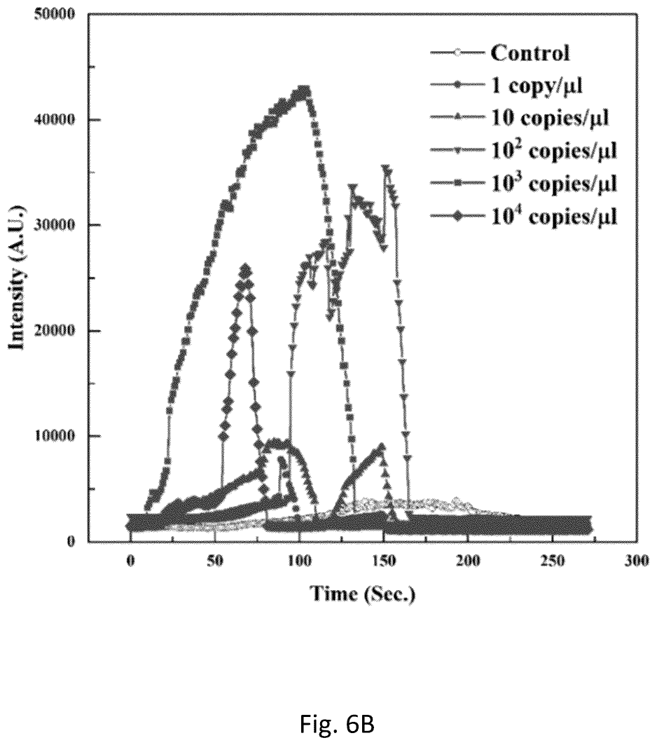

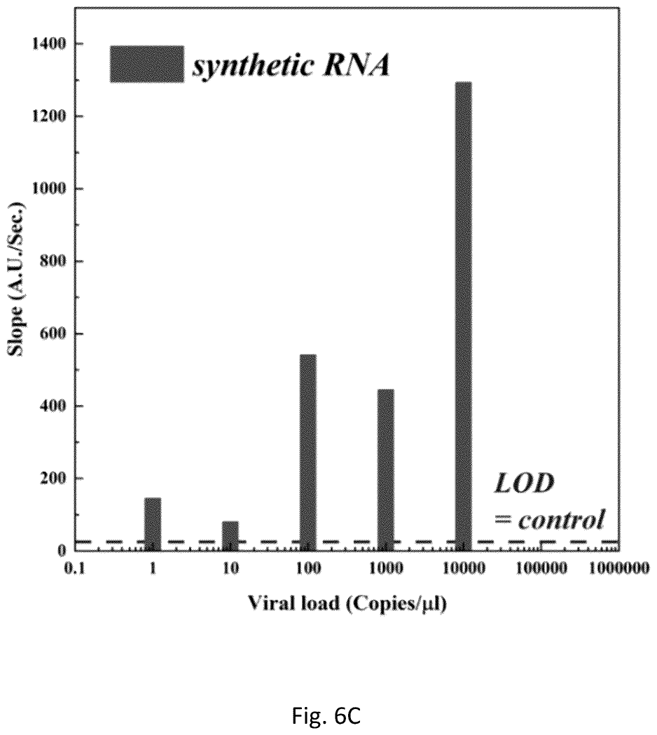

The detection platform was applied to the detection of SARS-COV-2 RNA without prior reverse transcription. To demonstrate this capability, synthetic SARS-COV-2 was mixed with Cas 13a enzyme, a reaction buffer, the gRNA and the fluorescence reporter. The mixture was injected to the same concentrator chip and a voltage of 60 V was applied to across the microfluidic channel. Similar to the cDNA experiment, fluorescence signals were observed from all the five positive samples containing 1 to 104 copies/μl of synthetic SARS-COV-2 RNA while no significant signal was observed from the negative control sample. For example, a clearly visible fluorescence signal of 10³ copies/μl started to show at 1 minute after applying voltage across the microfluidic channel, indicating the rapid detection of SARS-COV-2 RNA in our integrated microfluidic chip (FIG. 6A) without performing any prior PCR amplification.

The intensity plot in FIG. 6B shows that all the positive RNA samples from 1 copy/μl to 10⁴ copies/μl can clearly be detected within 2 minutes and the background signal intensity from the negative control was negligible compared to the signal intensity from the positive samples. In addition, the slope by linear regression clearly distinguished between the negative control and positive samples, demonstrating that the amplification-free detection platform was capable to detecting SARS-COV-2 RNA without prior amplification as well (FIG. 6C).

These results demonstrate amplification-free detection of SARS-COV-2 RNA by using the ICP integrated microfluidic chip. By eliminating sample preparation steps such as reverse transcription and amplification of RNA, the rapid detection of synthetic SARS-COV-2 RNA could be achieved within less than 5 minutes. Changing the composition and reaction ratio between Cas13a enzyme and gRNA could provide improved detection. Controlling these reaction parameters could enable a quantitative detection on our biosensing platform. This rapid amplification-free detection platform is highly promising for point care of diagnostics.

One Step CRISPR-Cas Detection of SARS-COV-2 cDNA from Patient Samples by Using the Microfluidic Concentrator Chip with Ion Concentration Polarization (ICP)

Twelve positive patient samples and five healthy subject samples were tested using the integrated microfluidic chip with ICP. SARS-COV-2 RNAs from the patient samples were extracted using the conventional RNA extraction kit by the collaborating group and provided to us for testing. cDNA was synthesized by reverse transcription.

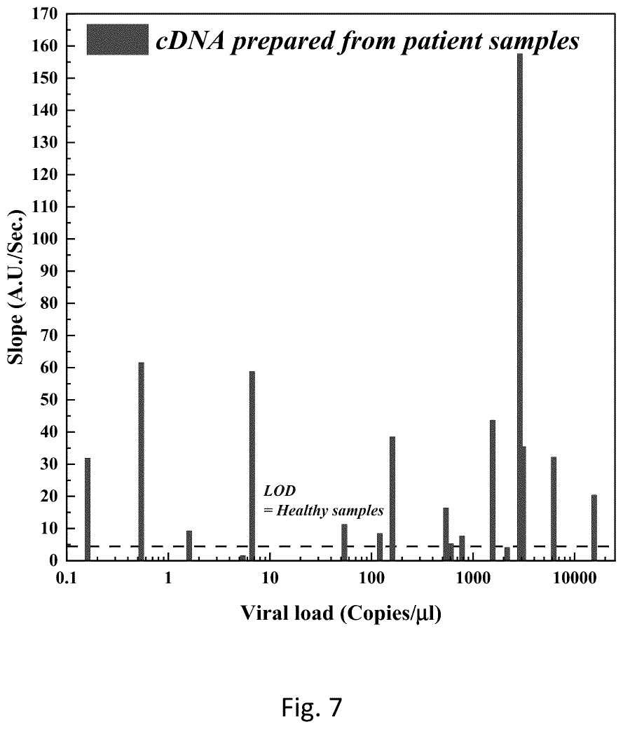

As shown in the slope graph obtained by linear regression of intensity from CRISPR-Cas12a detection (FIG. 7), this approach detected 8 of 12 positive patient samples with various copy numbers down to approximately 100 copies/μl, as recommended by CDC. This approach reduces the detection time dramatically comparted to other conventional approaches such as PCR or RT-LAMP which requires amplification process.

### An Amplification-Free Detection of Nucleic Acids by CRISPR/Cas in an Electrokinetic Microfluidic Concentrator Chip

One of the fundamental significances of the detection process lies in ensuring reproducibility. However, it is crucial to acknowledge that RNA, being inherently susceptible to degradation, is highly vulnerable to the activity of ribonucleases (RNases) present throughout the entire workflow, spanning from sample preparation to the subsequent detection steps. In order to minimize the potentially exist threaten of RNA degradation, the inclusion of an RNase Inhibitor would be essential.

To effectively demonstrate this, a stock solution of RNase Inhibitor (Murine) was simply mixed into the premixture of detection components, resulting in the master mix with the final concentration of 1 U/μL of the RNase Inhibitor. The premixture of detection comprises pivotal elements such as Cas13a, a reaction buffer, the guide RNA, and the fluorescent reporter reagents.

Subsequently, the master mix of the control or the seven positive samples, ranging from 10-1 to 105 copies/μL, was individually injected into the concentrator chip. A voltage of 60 V was applied across the microfluidic channel. In order to confirm reproducibility, triplicates were performed for all the samples, both for the control and the seven positive samples. The results were then quantitatively analyzed by determining maximum intensity and the initial velocity through linear regression.

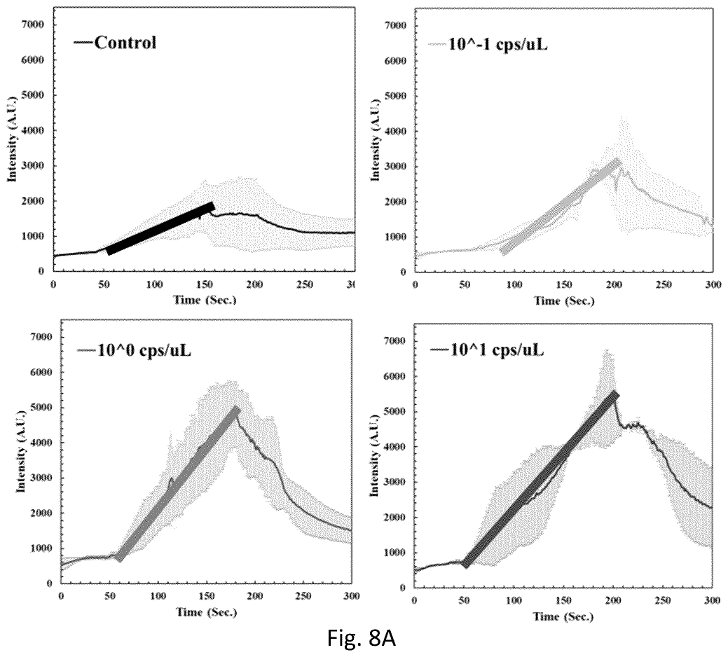

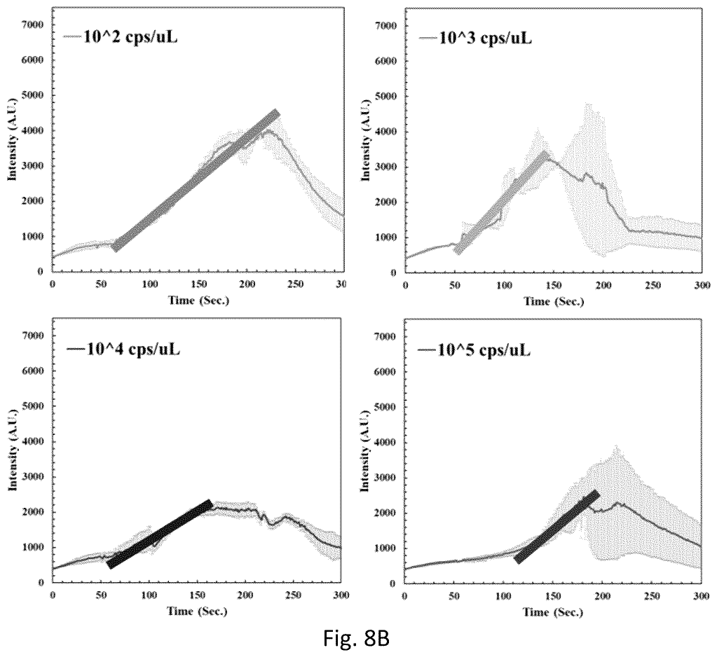

As expected, clear fluorescence signals were observed from all the seven positive samples ranging from 10-1 to 105 copies/μL of the SARS-COV-2 RNA, while relatively weak signal was observed from the negative control sample. The intensity plots in FIGS. 8A and 8B show that all the positive RNA samples from 10-1 to 105 copies/μL started to show remarkable intensity after approximately 60 seconds from applying voltage, and can clearly be detected within 2-3 minutes. Although, there is weak background signal generated from the negative control, the intensity is much less than the others from all positive samples.

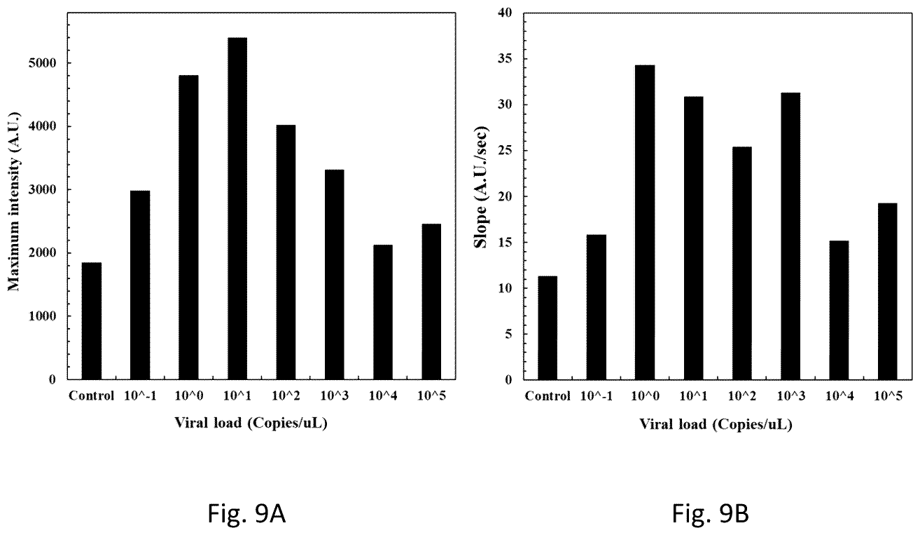

To further quantify the result, maximum intensity was obtained from the quantitative intensity and the initial velocity was calculated by linear regression after the lag phase, as indicated by bold line in each graph (see plots in FIGS. 8A and 8B). As shown in the maximum intensity and the initial velocity in FIGS. 9A and 9B, the negative control and positive samples were clearly distinguished from one another, demonstrating the disclosed amplification-free detection platform was capable of detecting SARS-COV-2 RNA without prior amplification as well.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A microfluidic concentrator chip device comprising:
a microfluidic chip comprising a flat body comprising a top and bottom surface, a first end, a second end, and a length therebetween;
a channel in the body of the chip, extending from near the first end of the chip to near the second end of the chip, the channel comprising a bottom surface, a top surface, sidewalls extending from the bottom surface to the top surface, and a first end, a second end, and a length therebetween;
a hydrogel membrane having a height and a width positioned within the channel;
a buffer solution contained within the channel;
a first electrode positioned near the first end of the channel in fluid communication with the buffer solution;
a second electrode positioned near the second end of the channel in fluid communication with the buffer solution; and
a power supply connected to the first and second electrodes, configured to apply a voltage across the channel.

2. The device of claim 1, wherein the hydrogel membrane comprises a negatively charged sulfonate group.

3. The device of claim 1, wherein the hydrogel membrane comprises polymerized 2-Acrylamido-2-methylpropane sulfonic acid.

4. The device of claim 3, wherein the hydrogel membrane comprises N,N′-Methylenebisacrylamide as a crosslinker.

5. The device of claim 1, wherein the buffer comprises any of Cas12a, Cas13a, a reaction buffer, a ribonuclease inhibitor, and a fluorescent reporter reagent.

6. The device of claim 1, wherein the hydrogel membrane has a height that is between 25-40% of the height of the sidewalls of the channel.

7. The device of claim 1 wherein the device comprises one or more pillars extending downwards from the top surface of the channel, wherein the pillars pass through at least a portion of the hydrogel membrane, thereby anchoring the hydrogel membrane in the channel.

8. A method of detecting a target analyte in a sample on a microfluidic concentrator chip, the method comprising:
a) heating a test sample to a set temperature for a period of time;
b) flowing a test sample through a channel of a microfluidic concentrator chip wherein the test sample comprises a detectable molecule for detecting the presence of a target analyte in the test sample;
c) applying a direct current (DC) voltage across the chip to generate an ion concentration polarization (ICP) region on the microfluidic concentrator chip; and
d) detecting the detectable molecule in the ICP region, wherein an increase in the level of the detectable molecule in the ICP region as compared to a control level indicates the presence of the target molecule in the test sample.

9. The method of claim 8, wherein the test sample comprises a biological fluid sample selected from the group consisting of a saliva sample, a blood sample, a plasma sample and a serum sample.

10. The method of claim 8, comprising the steps of:
a) flowing a test sample through a channel of a microfluidic concentrator chip wherein the test sample comprises a Cas protein at least one gRNA molecule, a ribonuclease inhibitor and a detectable molecule for detecting the presence of a target nucleic acid molecule in the test sample;
b) applying a DC voltage across the chip to generate an ICP region on the microfluidic concentrator chip; and
c) detecting the detectable molecule in the ICP region, wherein an increase in the level of the detectable molecule in the ICP region as compared to a control level indicates the presence of a target nucleic acid molecule in the test sample.

11. The method of claim 10, wherein the Cas protein is selected from the group consisting of a Cas12a and Cas13a protein.

12. The method of claim 10, wherein the detectable molecule is a fluorophore quencher (FQ)-labeled reporter molecule.

13. The method of claim 10, wherein the gRNA molecule is specific for binding to a viral RNA molecule.

14. The method of claim 13, wherein the viral RNA molecule is a SARS-COV-2 viral RNA molecule, and further comprising the step of indicating a SARS-COV-2 viral infection in a subject when the target nucleic acid molecule is detected in the test sample.

15. A method of concentrating an analyte in a sample comprising:
a) introducing a fluid to an inlet of the channel in the microfluidic concentrator chip of claim 1; and
b) applying a DC voltage across the chip to generate an ICP region in the channel, until the analyte is concentrated within the ICP region.

16. The method of claim 15, further comprising the step of releasing the concentrated analyte containing fluid.

17. The method of claim 16, further comprising the step of applying the concentrated analyte containing fluid to a downstream assay for detection of the target analyte.

18. The method of claim 17, wherein the downstream assay is selected from the group consisting of an immunoassay, a microarray, DNA hybridization assay, morpholino assay, aptamer assay, peptide nucleic acid (PNA) assay, and a target analyte detection assay.

19. The method of claim 18, further comprising the step of applying the concentrated analyte containing fluid to a downstream detector for detection of the target analyte.

20. The method of claim 19, wherein the downstream detector is selected from the group selected from a fluorescence detector, an electrochemical detector, a mass spectrometer, optical microring resonator, surface plasmon resonance (SPR) detector and a conductivity detector.

21. The method of claim 15, wherein the target analyte is selected from the group consisting of a pathogen, a soluble antigen, a nucleic acid molecule, a toxin, a chemical, a bacterium, and a virus.
